(19) United States
(12) Patent Application Publication
(10) Pub. No.: US 2007/0088173 A1
(43) Pub. Date: Apr. 19, 2007

(54) Method for producing of nickel(0)/phosphorus ligand complexes

(75) Inventors: Gerd Haderlein, Grunstadt (DE); Robert Baumann, Mannheim (DE); Michael Bartsch, Neustadt (DE); Tim Jungkamp, Kapellen (BE); Hermann Luyken, Ludwigshafen (DE); Jens Scheidel, Hirschberg (DE); Wolfgang Siegel, Limburgerhof (DE)

Correspondence Address: CONNOLLY BOVE LODGE & HUTZ LLP, P.O. BOX 2207, WILMINGTON, DE, 19899-2207, US

(73) Assignee: Basf Aktiengesellschaft, Ludwigshafen (DE)

(21) Appl. No.: 10/577,681

(22) PCT Filed: Oct. 28, 2004
(86) PCT No.: PCT/EP04/12179

§ 371 Date: Apr. 28, 2006

(30) Foreign Application Priority Data
Oct. 30, 2003 (DE) 10351000.1

(51) Int. Cl. C07F 15/04; C07C 253/10

(52) U.S. Cl. 558/320; 556/13

(57) ABSTRACT

A process is described for preparing nickel(0)-phosphorus ligand complexes containing at least one nickel(0) central atom and at least one phosphorus ligand. The process comprises reducing a nickel(II) source comprising nickel bromide, nickel iodide or mixtures thereof in the presence of at least one phosphorus ligand.

The present invention relates to a process for preparing nickel(0)-phosphorus ligand complexes. The present invention further provides the mixtures which comprise nickel(0)-phosphorus ligand complexes and are obtainable by this process, and also relates to their use in the hydrocyanation of alkenes or isomerization of unsaturated nitrites.

Nickel complexes of phosphorus ligands are suitable catalysts for hydrocyanations of alkenes. For example, nickel complexes having monodentate phosphites are known which catalyze the hydrocyanation of butadiene to prepare a mixture of isomeric pentenenitriles. These catalysts are also suitable in a subsequent isomerization of the branched 2-methyl-3-butenenitrile to linear 3-pentenenitrile and the hydrocyanation of the 3-pentenenitrile to adiponitrile, an important intermediate in the preparation of nylon-6,6.

U.S. Pat. No. 3,903,120 describes the preparation of zerovalent nickel complexes having monodentate phosphite ligands starting from nickel powder. The phosphorus ligands have the general formula PZ₃ where Z is an alkyl, alkoxy or aryloxy group. In this process, finely divided elemental nickel is used. In addition, preference is given to carrying out the reaction in the presence of a nitrilic solvent and in the presence of an excess of ligand.

U.S. Pat. No. 3,846,461 describes a process for preparing zerovalent nickel complexes with triorganophosphite ligands by reacting triorganophosphite compounds with nickel chloride in the presence of a finely divided reducing agent which is more electropositive than nickel. The reaction according to U.S. Pat. No. 3,846,461 takes place in the presence of a promoter which is selected from the group consisting of NH₃, NH₄X, Zn(NH₃)₂X₂ and mixtures of NH₄X and ZnX₂, where X is a halide.

New developments have shown that it is advantageous to use nickel complexes having chelate ligands (multidentate ligands) in the hydrocyanation of alkenes, since these allow both higher activities and higher selectivities to be achieved coupled with increased on-stream time. The above-described prior art processes are not suitable for preparing nickel complexes having chelate ligands. However, the prior art also discloses processes which enable the preparation of nickel complexes having chelate ligands.

U.S. Pat. No. 5,523,453 describes a process for preparing nickel-containing hydrocyanation catalysts which contain bidentate phosphorus ligands. These complexes are prepared starting from soluble nickel(0) complexes by transcomplexing with chelate ligands. The starting compounds used are Ni(COD)₂ or (oTTP)₂Ni(C₂H₄) (COD=1,5-cyclooctadiene; oTTP═P(O-ortho-C₆H₄CH₃)₃). As a consequence of the complicated preparation of the starting nickel compounds, this process is expensive.

Alternatively, there is the possibility of preparing nickel(0) complexes starting from bivalent nickel compounds and chelate ligands by reduction. In this method, it is generally necessary to work at high temperatures, so that thermally unstable ligands in the complex in some cases decompose.

US 2003/0100442 A1 describes a process for preparing a nickel(0) chelate complex, in which nickel chloride is reduced in the presence of a chelate ligand and of a nitrilic solvent using a more electropositive metal than nickel, in particular zinc or iron. In order to achieve a high space-time yield, an excess of nickel is used which has to be removed again after the complexation. The process is generally carried out with aqueous nickel chloride, which may lead to its decomposition especially when hydrolyzable ligands are used. When operation is effected with anhydrous nickel chloride, especially when hydrolyzable ligands are used, it is essential according to US 2003/0100442 A1 that the nickel chloride is initially dried by a specific process in which very small particles having large surface area and therefore high reactivity are obtained. A particular disadvantage of the process is that this fine nickel chloride dust prepared by spray drying is carcinogenic. A further disadvantage of this process is that operation is generally effected at elevated reaction temperatures, which may lead to decomposition of the ligands or of the complex especially in the case of thermally unstable ligands. It is a further disadvantage that operation has to be effected with an excess of reagents, in order to achieve economically viable conversions. These excesses have to be removed in a costly and inconvenient manner on completion of the reaction and optionally recycled.

GB 1 000 477 and BE 621 207 relate to processes for preparing nickel(0) complexes by reducing nickel(II) compounds using phosphorus ligands.

U.S. Pat. No. 4,385,007 describes a process for preparing nickel(0) complexes which are used as catalysts in combination with organoborane as a promoter for preparation of dinitriles. In this process, the catalyst and the promoter are obtained from a catalyst-active composition which has already been used in the preparation of adiponitrile by hydrocyanating pentenenitriles.

U.S. Pat. No. 3,859,327 describes a process for preparing nickel(0) complexes which are used as catalysts in combination with zinc chloride as a promoter for the hydrocyanation of pentenenitriles. In this process, nickel sources are used which stem from hydrocyanation reactions.

It is an object of the present invention to provide a process for preparing nickel(0) complexes having phosphorus ligands which substantially avoids the above-described disadvantages of the prior art. In particular, an anhydrous nickel source should be used, so that hydrolyzable ligands are not decomposed during the complexation. In addition, the reaction conditions should be gentle, so that thermally unstable ligands and the resulting complexes do not decompose. In addition, the process according to the invention should preferably enable the use of only a slight excess, if any, of the reagents, so that there is, if at all possible, no need to remove these substances after the complex has been prepared. The process should also be suitable for preparing nickel(0)-phosphorus ligand complexes having chelate ligands.

We have found that this object is achieved by a process for preparing nickel(0)-phosphorus ligand complexes which contain at least one nickel(0) central atom and at least one phosphorus ligand.

In the process according to the invention, a nickel(II) source comprising nickel bromide, nickel iodide or mixtures thereof is reduced in the presence of at least one phosphorus ligand.

It has been found in accordance with the invention that the nickel halides, nickel bromide and nickel iodide, unlike nickel chloride, may be used in complexation reactions to prepare nickel(0) complexes without the spray drying described in US 2003/0100442 A1. This makes a complicated drying process, as is necessary for nickel chloride, superfluous, since the reactivity of the nickel sources used in accordance with the invention is achieved irrespective of the crystal size.

In a particular embodiment of the present invention, the process according to the invention is thus carried out without preceding special drying, in particular without preceding spray drying of the nickel(II) source.

In the process according to the invention, nickel bromide and nickel iodide may each be used as the anhydrate or hydrate. In the context of the present invention, a hydrate of nickel bromide or iodide refers to a di- or hexahydrate or an aqueous solution. Preference is given to using anhydrates of nickel bromide or iodide, in order to substantially avoid hydrolyzing the ligand.

The process according to the invention is preferably carried out in the presence of a solvent. The solvent is selected in particular from the group consisting of organic nitrites, aromatic hydrocarbons, aliphatic hydrocarbons and mixtures of the aforementioned solvents. With regard to the organic nitrites, preference is given to acetonitrile, propionitrile, n-butyronitrile, n-valeronitrile, cyanocyclopropane, acrylonitrile, crotonitrile, allyl cyanide, cis-2-pentenenitrile, trans-2-pentenenitrile, cis-3-pentenenitrile, trans-3-pentenenitrile, 4-pentenenitrile, 2-methyl-3-butenenitrile, Z-2-methyl-2-butenenitrile, E-2-methyl-2-butenenitrile, ethylsuccinonitrile, adiponitrile, methylglutaronitrile or mixtures thereof. With regard to the aromatic hydrocarbons, benzene, toluene, o-xylene, m-xylene, p-xylene or mixtures thereof may preferably be used. Aliphatic hydrocarbons may preferably be selected from the group of the linear or branched aliphatic hydrocarbons, more preferably from the group of the cycloaliphatics, such as cyclohexane or methylcyclohexane, or mixtures thereof. Particular preference is given to using cis-3-pentenenitrile, trans-3-pentenenitrile, adiponitrile, methylglutaronitrile or mixtures thereof as the solvent.

Preference is given to using an inert solvent.

The concentration of the solvent is preferably from 10 to 90% by mass, more preferably from 20 to 70% by mass, in particular from 30 to 60% by mass, based in each case on the finished reaction mixture.

Ligands

In the process according to the invention, phosphorus ligands are used which are preferably selected from the group consisting of mono- or bidentate phosphines, phosphites, phosphinites and phosphonites.

These phosphorus ligands preferably have the formula I
P(X¹R¹)(X²R²)(X³R³) (I).
In the context of the present invention, compound I is a single compound or a mixture of different compounds of the aforementioned formula.

According to the invention, X¹, X², X³ each independently are oxygen or a single bond. When all of the X¹, X² and X³ groups are single bonds, compound I is a phosphine of the formula P(R¹R², R³) with the definitions of R¹, R² and R³ specified in this description.

When two of the X¹, X² and X³ groups are single bonds and one is oxygen, compound I is a phosphinite of the formula P(OR¹)(R²)(R³) or P(R¹)(OR²)(R³) or P(R¹)(R²)(OR³) with the definitions of R¹, R² and R³ specified below.

When one of the X¹, X² and X³ groups is a single bond and two are oxygen, compound I is a phosphonite of the formula P(OR¹)(OR²)(R³) or P(R¹)(OR²)(OR³) or P(OR¹)(R²)(OR³) with the definitions of R¹, R² and R³ specified in this description.

In a preferred embodiment, all X¹, X² and X³ groups should be oxygen, so that compound I is advantageously a phosphite of the formula P(OR¹)(OR²)(OR³) with the definitions of R¹, R² and R³ specified below.

According to the invention, R¹, R², R³ are each independently identical or different organic radicals. R¹, R² and R³ are each independently alkyl radicals preferably having from 1 to 10 carbon atoms, such as methyl, ethyl, n-propyl, i-propyl, n-butyl, i-butyl, s-butyl, t-butyl, aryl groups such as phenyl, o-tolyl, m-tolyl, p-tolyl, 1-naphthyl, 2-naphthyl, or hydrocarbyl, preferably having from 1 to 20 carbon atoms, such as 1,1′-biphenol, 1,1′-binaphthol. The R¹, R² and R³ groups may be bonded together directly, i.e. not solely via the central phosphorus atom. Preference is given to the R¹, R² and R³ groups not being bonded together directly.

In a preferred embodiment, R¹, R² and R³ are radicals selected from the group consisting of phenyl, o-tolyl, m-tolyl and p-tolyl. In a particularly preferred embodiment, a maximum of two of the R¹, R² and R³ groups should be phenyl groups.

In another preferred embodiment, a maximum of two of the R¹, R² and R³ groups should be o-tolyl groups.

Particularly preferred compounds I which may be used are those of the formula Ia
(o-tolyl-O—)_{w}(m-tolyl-O—)ₓ(p-tolyl-O—)_{y}(phenyl-O—)_{z}P (Ia)
where w, x, y, z are each a natural number, and the following conditions apply: w+x+y+z=3 and w, z≦2.

Such compounds Ia are, for example, (p-tolyl-O—)(phenyl-O—)₂P, (m-tolyl-O—)(phenyl-O—)₂P, (o-tolyl-O—)(phenyl-O—)₂P, (p-tolyl-O—)₂(phenyl-O—)P, (m-tolyl-O—)₂(phenyl-O—)P, (o-tolyl-O—)₂(phenyl-O—)P, (m-tolyl-O—)(p-tolyl-O—)(phenyl-O—)P, (o-tolyl-O—)(p-tolyl-O—)(phenyl-O—)P, (o-tolyl-O—)(m-tolyl-O—)(phenyl-O—)P, (p-tolyl-O—)₃P, (m-tolyl-O—)(p-tolyl-O—)₂P, (o-tolyl-O—)(p-tolyl-O—)₂P, (m-tolyl-O—)₂(p-tolyl-O—)P, (o-tolyl-O—)₂(p-tolyl-O—)P, (o-tolyl-O—)(m-tolyl-O—)(p-tolyl-O—)P, (m-tolyl-O—)₃P, (o-tolyl-O—)(m-tolyl-O—)₂P (o-tolyl-O—)₂(m-tolyl-O—)P or mixtures of such compounds.

Mixtures comprising (m-tolyl-O—)₃P, (m-tolyl-O—)₂(p-tolyl-O—)P, (m-tolyl-O—)(p-tolyl-O—)₂P and (P-tolyl-O—)₃P may be obtained for example by reacting a mixture comprising m-cresol and p-cresol, in particular in a molar ratio of 2:1, as obtained in the distillative workup of crude oil, with a phosphorus trihalide, such as phosphorus trichloride.

In another, likewise preferred embodiment, the phosphorus ligands are the phosphites, described in detail in DE-A 199 53 058, of the formula Ib:
P(O—R¹)ₓ(O—R²)_{y}(O—R³)_{z}(O—R⁴)ₚ (Ib)
where
R¹: aromatic radical having a C₁-C₁₈-alkyl substituent in the o-position to the oxygen atom which joins the phosphorus atom to the aromatic system, or having an aromatic substituent in the o-position to the oxygen atom which joins the phosphorus atom to the aromatic system, or having a fused aromatic system in the o-position to the oxygen atom which joins the phosphorus atom to the aromatic system,
R²: aromatic radical having a C₁-C₁₈-alkyl substituent in the m-position to the oxygen atom which joins the phosphorus atom to the aromatic system, or having an aromatic substituent in the m-position to the oxygen atom which joins the phosphorus atom to the aromatic system, or having a fused aromatic system in the m-position to the oxygen atom which joins the phosphorus atom to the aromatic system, the aromatic radical bearing a hydrogen atom in the o-position to the oxygen atom which joins the phosphorus atom to the aromatic system,
R³: aromatic radical having a C₁-C₁₈-alkyl substituent in the p-position to the oxygen atom which joins the phosphorus atom to the aromatic system, or having an aromatic substituent in the p-position to the oxygen atom which joins the phosphorus atom to the aromatic system, the aromatic radical bearing a hydrogen atom in the o-position to the oxygen atom which joins the phosphorus atom to the aromatic system,
R⁴: aromatic radical which bears substituents other than those defined for R¹, R² and R³ in the o-, m- and p-position to the oxygen atom which joins the phosphorus atom to the aromatic system, the aromatic radical bearing a hydrogen atom in the o-position to the oxygen atom which joins the phosphorus atom to the aromatic system,
x: 1 or 2,
y,z,p: each independently 0, 1 or 2, with the proviso that x+y+z+p=3.

Preferred phosphites of the formula Ib can be taken from DE-A 199 53 058. The R¹ radical may advantageously be o-tolyl, o-ethylphenyl, o-n-propylphenyl, o-isopropyl-phenyl, o-n-butylphenyl, o-sec-butylphenyl, o-tert-butylphenyl, (o-phenyl)phenyl or 1-naphthyl groups.

Preferred R² radicals are m-tolyl, m-ethylphenyl, m-n-propylphenyl, m-isopropylphenyl, m-n-butylphenyl, m-sec-butylphenyl, m-tert-butylphenyl, (m-phenyl)phenyl or 2-naphthyl groups.

Advantageous R³ radicals are p-tolyl, p-ethylphenyl, p-n-propylphenyl, p-isopropyl-phenyl, p-n-butylphenyl, p-sec-butylphenyl, p-tert-butylphenyl or (p-phenyl)phenyl groups.

The R⁴ radical is preferably phenyl. p is preferably zero. For the indices x, y, z and p in compound Ib, there are the following possibilities:

| x | y | z | p |
|---|---|---|---|
| 1 | 0 | 0 | 2 |
| 1 | 0 | 1 | 1 |
| 1 | 1 | 0 | 1 |
| 2 | 0 | 0 | 1 |
| 1 | 0 | 2 | 0 |
| 1 | 1 | 1 | 0 |
| 1 | 2 | 0 | 0 |
| 2 | 0 | 1 | 0 |
| 2 | 1 | 0 | 0 |

Preferred phosphites of the formula Ib are those in which p is zero, and R¹, R² and R³ are each independently selected from o-isopropylphenyl, m-tolyl and p-tolyl, and R⁴ is phenyl.

Particularly preferred phosphites of the formula Ib are those in which R¹ is the o-isopropylphenyl radical, R² is the m-tolyl radical and R³ is the p-tolyl radical with the indices specified in the table above; also those in which R¹ is the o-tolyl radical, R² is the m-tolyl radical and R³ is the p-tolyl radical with the indices specified in the table; additionally those in which R¹ is the 1-naphthyl radical, R² is the m-tolyl radical and R³ is the p-tolyl radical with the indices specified in the table; also those in which R¹ is the o-tolyl radical, R² is the 2-naphthyl radical and R³ is the p-tolyl radical with the indices specified in the table; and finally those in which R¹ is the o-isopropylphenyl radical, R² is the 2-naphthyl radical and R³ is the p-tolyl radical with the indices specified in the table; and also mixtures of these phosphites.

Phosphites of the formula Ib may be obtained by
a) reacting a phosphorus trihalide with an alcohol selected from the group consisting of R¹OH, R²OH, R³OH and R⁴OH or mixtures thereof to obtain a dihalophosphorous monoester,
b) reacting the dihalophosphorous monoester mentioned with an alcohol selected from the group consisting of R¹OH, R²OH, R³OH and R⁴OH or mixtures thereof to obtain a monohalophosphorous diester and
c) reacting the monohalophosphorous diester mentioned with an alcohol selected from the group consisting of R¹OH, R²OH, R³OH and R⁴OH or mixtures thereof to obtain a phosphite of the formula Ib.

The reaction may be carried out in three separate steps. Equally, two of the three steps may be combined, i.e. a) with b) or b) with c). Alternatively, all of steps a), b) and c) may be combined together.

Suitable parameters and amounts of the alcohols selected from the group consisting of R¹OH, R²OH, R³OH and R⁴OH or mixtures thereof may be determined readily by a few simple preliminary experiments.

Useful phosphorus trihalides are in principle all phosphorus trihalides, preferably those in which the halide used is Cl, Br, I, in particular Cl, and mixtures thereof. It is also possible to use mixtures of various identically or differently halogen-substituted phosphines as the phosphorus trihalide. Particular preference is given to PCl₃. Further details on the reaction conditions in the preparation of the phosphites Ib and for the workup can be taken from DE-A 199 53 058.

The phosphites Ib may also be used in the form of a mixture of different phosphites Ib as a ligand. Such a mixture may be obtained, for example, in the preparation of the phosphites Ib.

However, preference is given to the phosphorus ligand being multidentate, in particular bidentate. The ligand used therefore preferably has the formula II 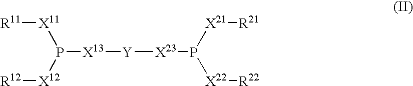
where
X¹¹, X¹², X¹³ X²¹, X²², X²³ are each independently oxygen or a single bond
R¹¹, R¹² are each independently identical or different, individual or bridged organic radicals
R²¹, R²² are each independently identical or different, individual or bridged organic radicals,
Y is a bridging group.

In the context of the present invention, compound II is a single compound or a mixture of different compounds of the aforementioned formula.

In a preferred embodiment, X¹¹, X¹², X¹³, X²¹, X²², X²³ may each be oxygen. In such a case, the bridging group Y is bonded to phosphite groups.

In another preferred embodiment, X¹¹ and X¹² may each be oxygen and X¹³ a single bond, or X¹¹ and X¹³ each oxygen and X¹² a single bond, so that the phosphorus atom surrounded by X¹¹, X¹² and X¹³ is the central atom of a phosphonite. In such a case, X²¹, X²² and X²³ may each be oxygen, or X²¹ and X²² may each be oxygen and X²³ a single bond, or X²¹ and X²³ may each be oxygen and X²² a single bond, or X²³ may be oxygen and X²¹ and X²² each a single bond, or X²¹ may be oxygen and X²² and X²³ each a single bond, or X²¹, X²² and X²³ may each be a single bond, so that the phosphorus atom surrounded by X²¹, X²² and X²³ may be the central atom of a phosphite, phosphonite, phosphinite or phosphine, preferably a phosphonite.

In another preferred embodiment, X¹³ may be oxygen and X¹¹ and X¹² each a single bond, or X¹¹ may be oxygen and X¹² and X¹³ each a single bond, so that the phosphorus atom surrounded by X¹¹, X¹² and X¹³ is the central atom of a phosphonite. In such a case, X²¹, X²² and X²³ may each be oxygen, or X²³ may be oxygen and X²¹ and X²² each a single bond, or X²¹ may be oxygen and X²² and X²³ each a single bond, or X²¹, X²² and X²³ may each be a single bond, so that the phosphorus atom surrounded by X²¹, X²² and X²³ may be the central atom of a phosphite, phosphinite or phosphine, preferably a phosphinite.

In another preferred embodiment, X¹¹, X¹² and X¹³ may each be a single bond, so that the phosphorus atom surrounded by X¹¹, X¹² and X¹³ is the central atom of a phosphine. In such a case, X²¹, X²² and X²³ may each be oxygen, or X²¹, X²² and X²³ may each be a single bond, so that the phosphorus atom surrounded by X²¹, X²² and X²³ may be the central atom of a phosphite or phosphine, preferably a phosphine.

The bridging group Y is advantageously an aryl group which is substituted, for example by C₁-C₄-alkyl, halogen, such as fluorine, chlorine, bromine, halogenated alkyl, such as trifluoromethyl, aryl, such as phenyl, or is unsubstituted, preferably a group having from 6 to 20 carbon atoms in the aromatic system, in particular pyrocatechol, bis(phenol) or bis(naphthol).

The R¹¹ and R¹² radicals may each independently be identical or different organic radicals. Advantageous R¹¹ and R¹² radicals are aryl radicals, preferably those having from 6 to 10 carbon atoms, which may be unsubstituted or mono- or polysubstituted, in particular by C₁-C₄-alkyl, halogen, such as fluorine, chlorine, bromine, halogenated alkyl, such as trifluoromethyl, aryl, such as phenyl, or unsubstituted aryl groups.

The R²¹ and R²² radicals may each independently be the same or different organic radicals. Advantageous R²¹ and R²² radicals are aryl radicals, preferably those having from 6 to 10 carbon atoms, which may be unsubstituted or mono- or polysubstituted, in particular by C₁-C₄-alkyl, halogen, such as fluorine, chlorine, bromine, halogenated alkyl, such as trifluoromethyl, aryl, such as phenyl, or unsubstituted aryl groups.

The R¹¹ and R¹² radicals may each be separate or bridged. The R²¹ and R²² radicals may also each be separate or bridged. The R¹¹, R¹², R²¹ and R²² radicals may each be separate, two may be bridged and two separate, or all four may be bridged, in the manner described.

In a particularly preferred embodiment, useful compounds are those of the formula I, II, III, IV and V specified in U.S. Pat. No. 5,723,641. In a particularly preferred embodiment, useful compounds are those of the formula I, II, III, IV, V, VI and VII specified in U.S. Pat. No. 5,512,696, in particular the compounds used there in examples 1 to 31. In a particularly preferred embodiment, useful compounds are those of the formula I, II, III, IV, V, VI, VII, VIII, IX, X, XI, XII, XIII, XIV and XV specified in U.S. Pat. No. 5,821,378, in particular the compounds used there in examples 1 to 73.

In a particularly preferred embodiment, useful compounds are those of the formula I, II, III, IV, V and VI specified in U.S. Pat. No. 5,512,695, in particular the compounds used there in examples 1 to 6. In a particularly preferred embodiment, useful compounds are those of the formula I, II, III, IV, V, VI, VII, VIII, IX, X, XI, XII, XIII and XIV specified in U.S. Pat. No. 5,981,772, in particular the compounds used there in examples 1 to 66.

In a particularly preferred embodiment, useful compounds are those specified in U.S. Pat. No. 6,127,567 and the compounds used there in examples 1 to 29. In a particularly preferred embodiment, useful compounds are those of the formula I, II, III, IV, V, VI, VII, VIII, IX and X specified in U.S. Pat. No. 6,020,516, in particular the compounds used there in examples 1 to 33. In a particularly preferred embodiment, useful compounds are those specified in U.S. Pat. No. 5,959,135, and the compounds used there in examples 1 to 13.

In a particularly preferred embodiment, useful compounds are those of the formula I, II and III specified in U.S. Pat. No. 5,847,191. In a particularly preferred embodiment, useful compounds are those specified in U.S. Pat. No. 5,523,453, in particular the compounds illustrated there in formula 1, 2, 3, 4, 5, 6, 7, 8, 9, 10, 11, 12, 13, 14, 15, 16, 17, 18, 19, 20 and 21. In a particularly preferred embodiment, useful compounds are those specified in WO 01/14392, preferably the compounds illustrated there in formula V, VI, VII, VIII, IX, X, XI, XII, XIII, XIV, XV, XVI, XVII, XXI, XXII, XXIII.

In a particularly preferred embodiment, useful compounds are those specified in WO 98/27054. In a particularly preferred embodiment, useful compounds are those specified in WO 99/13983. In a particularly preferred embodiment, useful compounds are those specified in WO 99/64155.

In a particularly preferred embodiment, useful compounds are those specified in the German patent application DE 100 380 37. In a particularly preferred embodiment, useful compounds are those specified in the German patent application DE 100 460 25. In a particularly preferred embodiment, useful compounds are those specified in the German patent application DE 101 502 85.

In a particularly preferred embodiment, useful compounds are those specified in the German patent application DE 101 502 86. In a particularly preferred embodiment, useful compounds are those specified in the German patent application DE 102 071 65. In a further particularly preferred embodiment of the present invention, useful phosphorus chelate ligands are those specified in US 2003/0100442 A1.

In a further particularly preferred embodiment of the present invention, useful phosphorus chelate ligands are those specified in the German patent application of reference number DE 103 50 999.2 of Oct. 30, 2003 which has an earlier priority date but had not been published at the priority date of the present application.

The compounds I, Ia, Ib and II described and their preparation are known per se. The phosphorus ligands used may also be mixtures comprising at least two of the compounds I, Ia, Ib and II.

In a particularly preferred embodiment of the process according to the invention, the phosphorus ligand of the nickel(0) complex and/or the free phosphorus ligand is selected from tritolyl phosphite, bidentate phosphorus chelate ligands and the phosphites of the formula Ib
P(O—R¹)ₓ(O—R²)_{y}(O—R³)_{z}(O—R⁴)ₚ (Ib)
where R¹, R² and R³ are each independently selected from o-isopropylphenyl, m-tolyl and p-tolyl, R⁴ is phenyl; x is 1 or 2, and y, z, p are each independently 0, 1 or 2 with the proviso that x+y+z+p=3; and mixtures thereof.

In the process according to the invention, the concentration of the ligand in the solvent is preferably from 1 to 90% by weight, more preferably from 5 to 80% by weight, in particular from 50 to 80% by weight.

The reducing agent used in the process according to the invention is preferably selected from the group consisting of metals which are more electropositive than nickel, metal alkyls, electrical current, complex hydrides and hydrogen.

When the reducing agent in the process according to the invention is a metal which is more electropositive than nickel, this metal is preferably selected from the group consisting of sodium, lithium, potassium, magnesium, calcium, barium, strontium, titanium, vanadium, iron, cobalt, copper, zinc, cadmium, aluminum, gallium, indium, tin, lead and thorium. Particular preference is given in this context to iron and zinc. When aluminum is used as the reducing agent, it is advantageous when it is preactivated by reaction with a catalytic amount of mercury(II) salt or metal alkyl. Preference is given to using triethylaluminum for the preactivation in an amount of preferably from 0.05 to 50 mol %, more preferably from 0.5 to 10 mol %. The reduction metal is preferably finely divided, the expression “finely divided” meaning that the metal is used in a particle size of less than 10 mesh, more preferably less than 20 mesh.

When the reducing agent used in the process according to the invention is a metal which is more electropositive than nickel, the amount of metal is preferably from 0.1 to 5% by weight, based on the reaction mixture.

When metal alkyls are used as reducing agents in the process according to the invention, they are preferably lithium alkyls, sodium alkyls, magnesium alkyls, in particular Grignard reagents, zinc alkyls or aluminum alkyls. Particular preference is given to aluminum alkyls such as trimethylaluminum, triethylaluminum, triisopropylaluminum or mixtures thereof, in particular triethylaluminum. The metal alkyls may be used without solvent or dissolved in an inert organic solvent such as hexane, heptane or toluene.

When complex hydrides are used as the reducing agent in the process according to the invention, preference is given to using metal aluminum hydrides such as lithium aluminum hydride, or metal borohydrides such as sodium borohydride.

The molar ratio of redox equivalents between the nickel(II) source and the reducing agent is preferably from 1:1 to 1:100, more preferably from 1:1 to 1:50, in particular from 1:1 to 1:5.

In the process according to the invention, the ligand to be used may also be present in a ligand solution which has already been used as a catalyst solution in hydrocyanation reactions and which is depleted of nickel(0). This residual catalyst solution generally has the following composition:
from 2 to 60% by weight, in particular from 10 to 40% by weight, of pentenenitriles,
from 0 to 60% by weight, in particular from 0 to 40% by weight, of adiponitrile,
from 0 to 10% by weight, in particular from 0 to 5% by weight, of other nitriles,
from 10 to 90% by weight, in particular from 50 to 90% by weight, of phosphorus ligand and
from 0 to 2% by weight, in particular from 0 to 1% by weight, of nickel(0).

In the process according to the invention, the free ligand present in the residual catalyst solution may thus be converted back to a nickel(0) complex.

In a particular embodiment of the present invention, the ratio of the nickel(II) source to phosphorus ligand is from 1:1 to 1:100. Further preferred ratios of nickel(II) source to phosphorus ligand are from 1:1 to 1:3, in particular from 1:1 to 1:2.

The process according to the invention may preferably be carried out in such a way that unreacted nickel bromide or iodide may be removed after the complex synthesis and recycled to the preparation of the complexes. Unreacted nickel bromide or iodide may be removed by processes known to those skilled in the art such as filtration, centrifugation, sedimentation or using hydrocyclones, as described, for example, in Ullmann's Encyclopedia of Industrial Chemistry, Unit Operation I, Vol. B2, VCH, Weinheim, 1988, in chapter 10, pages 10-1 to 10-59, chapter 11, pages 11-1 to 11-27 and chapter 12, pages 12-1 to 12-61.

The process according to the invention may be carried out at any pressure. For practical reasons, preference is given to pressures between 0.1 bar abs and 5 bar abs, preferably 0.5 bar abs and 1.5 bar abs.

The process according to the invention is preferably carried out under inert gas, for example argon or nitrogen.

The process according to the invention may be carried out in batch mode or continuously.

In a particular embodiment of the present invention, the process according to the invention comprises the following process steps:
(1) preparing a solution or suspension of the nickel(II) source comprising nickel bromide, nickel iodide and a mixture thereof in a solvent under inert gas,
(2) stirring the solution or suspension stemming from process step (1) at a precomplexation temperature of from 20 to 120° C. and for a precomplexation period of from 1 minute to 24 hours,
(3) adding at least one reducing agent to the solution or suspension stemming from process step (2) at an addition temperature of from 20 to 120° C.,
(4) stirring the solution or suspension stemming from process step (3) for a reaction period of from 30 minutes to 24 hours at a reaction temperature of from 20 to 120° C.

The precomplexation temperatures, addition temperatures, and reaction temperatures may each independently be from 20° C. to 120° C. In the precomplexation, addition and reaction, particular preference is given to temperatures of from 30° C. to 80° C.

The precomplexation periods, addition periods and reaction periods may each independently be from 1 minute to 24 hours. The precomplexation period is in particular from 1 minute to 3 hours. The addition period is preferably from 1 minute to 30 minutes. The reaction period is preferably from 20 minutes to 5 hours.

The process according to the invention has the advantage of a high reactivity of the nickel bromide or nickel iodide. A complicated drying process, as is necessary for nickel chloride according to US 2003/0100442 A1, is redundant, since the reactivity of the nickel sources used in accordance with the invention is achieved irrespective of the crystal size. This makes reaction possible even at low temperatures. Moreover, it is not necessary to use an excess of nickel salt, as disclosed by the prior art. In addition, complete conversion with respect to the nickel(II) bromide or nickel(II) iodide and the reducing agent may be achieved, which makes its subsequent removal superfluous. As a consequence of the high reactivity, nickel: ligand ratios of up to 1:1 may be obtained.

The present invention further provides the solutions comprising nickel(0)-phosphorus ligand complexes obtainable by the process according to the invention, and also their use in the hydrocyanation of alkenes, in particular in the hydrocyanation of butadiene to prepare a mixture of pentenenitriles, the isomerization of 2-methyl-3-butenenitrile to 3-pentenenitrile, and the renewed hydrocyanation of 3-pentenenitrile to adiponitrile which follows in the synthesis of adiponitrile.

The present inventions are illustrated by the examples which follow.

### EXAMPLES

In the examples of complex synthesis, the chelate ligand solution used was a solution of the chelate phosphonite 1 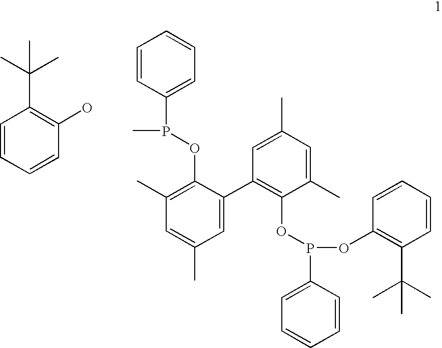
in 3-pentenenitrile (65% by weight of chelate, 35% by weight of 3-pentenenitrile).

To determine the conversion, the complex solutions prepared were investigated for their content of active, complexed Ni(0). To this end, the solutions were admixed with tri(m/p-tolyl) phosphite (typically 1 g of phosphite per 1 g of solution) and kept at 80° C. for approx. 30 min in order to achieve complete transcomplexation. Subsequently, the current-voltage curve for the electrochemical oxidation was determined in a cyclic voltammetry measurement apparatus in unstirred solution against a reference electrode, which provides the peak current which is proportional to the concentration and determines, via calibration with solutions of known Ni(0) concentration, the Ni(0) content of the test solutions, corrected by the subsequent dilution with tri(m/p-tolyl) phosphite. The Ni(0) values quoted in the examples report the content of Ni(0) in % by weight based on the entire reaction solution, determined by this method.

In Examples 1 to 6, the nickel source used was NiBr₂ and the reducing agent used was zinc powder:

### Example 1

In a 500 ml flask with stirrer, 18.6 g (85 mmol) of NibR₂ were suspended under argon in 13 g of 3-pentenenitrile, 100 g of chelate solution (86 mmol of ligand) were added and the mixture was stirred at 80° C. for 10 min. After cooling to 50° C., 8 g of Zn powder (122 mmol, 14 eq.) were added and the mixture was stirred at 50° C. for 3 h. An Ni(0) value of 1.2% (33% conversion) was measured.

### Example 2

A reaction was carried out in a similar manner to Example 1, except that the temperature was reduced to 40° C. before the Zn powder was added. After 5 h, an Ni(0) value of 2.0% (56% conversion) was measured.

### Example 3

A reaction was carried out in a similar manner to Example 1, except that the temperature was reduced to 30° C. before the Zn powder was added. After 12 h, an Ni(0) value of 1.3% (36% conversion) was measured.

### Example 4

A reaction was carried out in a similar manner to Example 1, except that 61 g of 3-pentenenitrile were used to dilute the reaction mixture, and the temperature was reduced to 60° C. before the Zn powder was added. After 4 h, an Ni(0) value of 1.6% (60% conversion) was measured.

### Example 5

In a 500 ml flask with stirrer, 14 g (64 mmol) of NiBr₂ were suspended under argon in 13 g of 3-pentenenitrile, 100 g of chelate solution (86 mmol of ligand) were added and the mixture was stirred at 80° C. for 10 min. After cooling to 50° C., 6 g of Zn powder (92 mmol, 1.4 eq.) were added and the mixture was stirred at 50° C. for 5 h. An Ni(0) value of 1.2% (43% conversion) was measured.

### Example 6

In a 500 ml flask with stirrer, 9.3 g (43 mmol) of NiBr₂ were suspended under argon in 13 g of 3-pentenenitrile, 100 g of chelate solution (86 mmol of ligand) were added and the mixture was stirred at 80° C. for 10 min. After cooling to 50° C., 4 g of Zn powder (61 mmol, 1.4 eq.) were added and the mixture was stirred at 50° C. for 5 h. An Ni(0) value of 0.88% (44% conversion) was measured.

In Examples 7-10, the nickel source used was NiBr₂ and the reducing agent used was iron powder.

### Example 7

In a 500 ml flask with stirrer, 18.6 g (85 mmol) of NiBr₂ were suspended under argon in 13 g of 3-pentenenitrile, 100 g of chelate solution (86 mmol of ligand) were added and the mixture was stirred at 80° C. for 10 min. After cooling to 30° C., 5.3 g of Fe powder (95 mmol, 1.1 eq.) were added and the mixture was stirred at 30° C. for 4 h. An Ni(0) value of 0.75% (42% conversion) was measured.

### Example 8

A reaction was carried out in a similar manner to Example 7, except that the temperature was reduced to 40° C. before the Fe powder was added. After 4 h, an Ni(0) value of 0.8% (44% conversion) was measured.

### Example 9

A reaction was carried out in a similar manner to Example 7, except that the temperature was reduced to 60° C. before the Fe powder was added. After 4 h, an Ni(0) value of 1.0% (56% conversion) was measured.

### Example 10

A reaction was carried out in a similar manner to Example 7, except that the temperature was kept at 80° C. before the Fe powder was added. After 4.5 h, an Ni(0) value of 1.6% (89% conversion) was measured.

In the example which follows, the reducing agent used was aluminum powder preactivated with Et3 μl.

### Example 11

In a 250 ml flask with stirrer, 18 g (82 mmol) of NiBr₂ were dissolved under argon in 13 g of 3-pentenenitrile, admixed with 3.2 g (119 mmol) of aluminum powder, 3 ml of a 1 M solution of triethylaluminum in hexane (3 mmol) were added and the mixture was stirred at room temperature for 30 min to activate the aluminum powder. Subsequently, 100 g of chelate solution (86 mmol of ligand) were added and the mixture was stirred at 80° C. for 3 h. An Ni(0) value of 0.8% (25% conversion) was measured.

In Examples 12 and 13, the reducing agent used was Et3Al.

### Example 12

In a 250 ml flask with stirrer, 6.3 g (29 mmol) of NiBr₂ were suspended under argon in 67.3 g of chelate solution (58 mmol of ligand) and cooled to 0° C. Subsequently, 20.1 of a 25% solution of triethylaluminum in toluene (44 mmol) were slowly metered in. After slowly warming the solution to room temperature, it was heated to 65° C. and stirred for another 4 h. An Ni(0) value of 0.9% (49% conversion) was measured.

### Example 13

In a 250 ml flask with stirrer, 6.3 g (29 mmol) of NiBr₂ were suspended under argon in 67.3 g of chelate solution (58 mmol of ligand). At 30° C., 25.1 of a 25% solution of triethylaluminum in toluene (55 mmol) were slowly metered in. Subsequently, the mixture was heated to 65° C. and stirred for 4 h. An Ni(0) value of 1.4% (81% conversion) was measured.

In Examples 14 and 15, the nickel salt used was nickel iodide.

### Example 14

In a 250 ml flask with stirrer, 27 g (86 mmol) of NiI₂ were dissolved under argon in 13 g of 3-pentenenitrile and 100 g of chelate solution (86 mmol of ligand) and stirred at 80° C. for 15 min. After cooling to 50° C., 7.2 g of Zn powder (110 mmol, 1.25 eq.) were added and the mixture was stirred at 50° C. for 4 h. An Ni(0) value of 2.2% (65% conversion) was measured.

### Example 15

A reaction was carried out in a similar manner to Example 12, except that the temperature was reduced to 30° C. before the Zn powder was added. After 4 h, an Ni(0) value of 0.5% (15% conversion) was measured.

In Examples 16 and 17, the ligand solution used was a residual catalyst solution which had already been used as the catalyst solution in hydrocyanation reactions and had been strongly depleted of Ni(0). The composition of the solution is approx. 20% by weight of pentenenitriles, approx. 6% by weight of adiponitrile, approx. 3% by weight of other nitriles, approx. 70% by weight of ligand (consisting of a mixture of 40 mol % of chelate phosphonite 1 and 60 mol % of tri(m/p-tolyl)phosphite) and a nickel(0) content of only 0.8% by weight.

### Example 16

In a 500 ml flask with stirrer, 18.6 g (85 mmol) of NiBr₂ were suspended under argon in 24 g of 3-pentenenitrile, admixed with 100 g of residual catalyst solution and stirred at 80° C. for 15 min. Subsequently, the mixture was cooled to 50° C., 8 g of Zn powder (122 mmol, 1.4 eq.) were added and the mixture was stirred at 50-55° C. for 5 h. An Ni(0) value of 1.9% (corresponding to an Ni:P ratio 1:4) was measured.

### Example 17

In a 500 ml flask with stirrer, 18.6 g (85 mmol) of NiBr₂ were suspended under argon in 24 g of 3-pentenenitrile, admixed with 100 g of residual catalyst solution and stirred at 80° C. for 20 min. Subsequently, 5.3 g of Fe powder (95 mmol, 1.1 eq.) were added at 80° C. and the mixture was stirred at 80° C. for 4.5 h. An Ni(0) value of 1.7% (corresponding to an Ni:P ratio=1:4.4) was measured.

In the comparative examples, commercially available, anhydrous nickel chloride was used as the nickel source:

### Comparative example 1

In a 500 ml flask with stirrer, 11 g (885 mmol) of NiCl₂ were suspended under argon in 13 g of 3-pentenenitrile, admixed with 100 g of chelate solution (86 mmol of ligand) and stirred at 80° C. for 15 min. After cooling to 40° C., 8 g of Zn powder (122 mmol, 1.4 eq.) were added and the mixture was stirred at 40° C. for 4 h. An Ni(0) value of 0.05% (1% conversion) was measured.

### Comparative example 2

A reaction was carried out in a similar manner to Comparative example 1, except that the temperature was kept at 80° C. when the Zn powder was added. After 5 h, an Ni(0) value of 0.4% (10% conversion) was measured.

### Comparative example 3

In a 500 ml flask with stirrer, 11 g (85 mmol) of NiCl₂ were suspended under argon in 13 g of 3-pentenenitrile, admixed with 100 g of chelate solution (86 mmol of ligand) and stirred at 80° C. for 15 min. After cooling to 60° C., 5.3 g of Zn powder (95 mmol, 1.1 eq.) were added and the mixture was stirred at 60-65° C. for 10 h. An Ni(0) value of 0.16% (4% conversion) was measured.

### Comparative example 4

A reaction was carried out in a similar manner to Comparative example 3, except that the temperature was kept at 80° C. when the Fe powder was added. After 10 h, an Ni(0) value of 0.4% (10% conversion) was measured.

1. A process for preparing a nickel(0)-phosphorus ligand complex containing at least one nickel(0) atom and at least one phosphorus ligand, which comprises reducing a nickel(II) source comprising nickel bromide, nickel iodide or mixtures thereof in the presence of at least one phosphorus ligand selected from the group of mono- or bidentate phosphonites, bidentate phosphites, bidentate phosphines and mono- or bidentate phosphinites.

2. The process according to claim 1, wherein reducing the nickel(II) source is conducted in the presences of a solvent selected from the group consisting of organic nitrites, aromatic or aliphatic hydrocarbons and mixtures thereof.

3. The process according to claim 2, wherein the concentration of the phosphorus ligand in the solvent is from 1 to 90% by weight, based on the solution.

4. The process according to claim 1, wherein reducing the nickel(II) source comprises metal reducing agents that are more electropositive than nickel.

5. The process according to claim 1, wherein reducing the nickel(II) source comprises reducing agents selected from the group consisting of metal alkyls, electrical current, complex hydrides and hydrogen.

6. The process according to claim 1, wherein the phosphorus ligand is obtained from a ligand solution that was used as a catalyst solution in hydrocyanation reactions.

7. The process according to claim 1, which comprises the following process steps:
(I) preparing a solution or suspension of nickel bromide, nickel iodide or a mixture thereof in a solvent under inert gas,
(II) stirring the solution or suspension prepared in step (I) at a precomplexation temperature of from 20 to 120° C. and for a precomplexation period of from 1 minute to 24 hours,
(III) adding at least one reducing agent to the solution or suspension prepared in step (II) at an addition temperature of from 20 to 120° C.,
(IV) stirring the suspension or solution from step (III) for a reaction period of from 20 minutes to 24 hours at a reaction temperature of from 20 to 120° C. and
(V) adding a chelate solution.

8. A mixture comprising a nickel(0)-phosphorus ligand complex, obtainable by a process according to claim 1.

9. The use of the mixtures comprising nickel(0)-phosphorus ligand complexes according to claim 8 in the hydrocyanation and isomerization of alkenes and in the hydrocyanation and isomerization of unsaturated nitrites.

10. The use of the mixtures comprising nickel(0)-phosphorus ligand complexes according to claim 7 in the hydrocyanation and isomerization of alkenes and in the hydrocyanation and isomerization of unsaturated nitrites.

11. A process for preparing a nickel(0)-phosphorus ligand complex comprising the following steps:
(i) preparing a solution or suspension of nickel bromide, nickel iodide or a mixture thereof in a solvent under inert gas, and stirring the nickel-containing solution or suspension at a precomplexation temperature of from 20 to 120° C. for a precomplexation period of from 1 minute to 24 hours; and
(ii) adding at least one reducing agent to the solution or suspension in step (i) at an addition temperature of from 20 to 120° C. and adding a chelate solution comprising at least one phosphorus ligand selected from the group of mono- or bidentate phosphonites, bidentate phosphites, bidentate phosphines and mono- or bidentate phosphinites; and
(iii) stirring the resulting suspension or solution from for a reaction period of from 20 minutes to 24 hours at a reaction temperature of from 20 to 120° C.

12. The process according to claim 11, wherein preparing the nickel-containing solution or suspension is conducted in the presence of a solvent selected from the group consisting of organic nitrites, aromatic or aliphatic hydrocarbons and mixtures thereof.

13. The process according to claim 11, wherein the phosphorus ligand is obtained from a ligand solution that was used as a catalyst solution in hydrocyanation reactions.

14. The process according to claim 1, wherein the phosphorus ligand is a bidentate phosphite of formula I

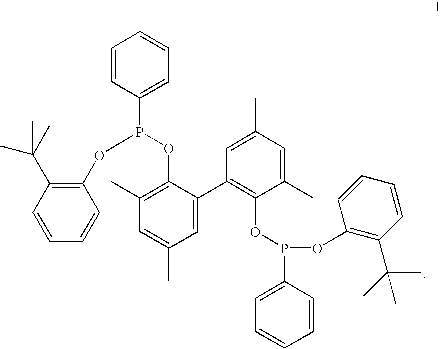

15. The process according to claim 11, wherein the phosphorus ligand is a bidentate phosphite of formula I

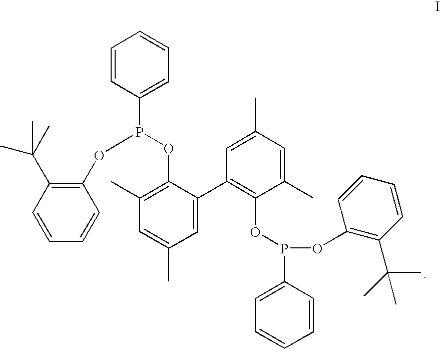

16. The process according to claim 1 wherein the nickel source is nickel bromide.

17. The process according to claim 11 wherein the nickel source is nickel bromide.

18. The process according to claim 1 wherein the nickel source is nickel iodide.

19. The process according to claim 11 wherein the nickel source is nickel iodide.
